(19) United States
(12) Patent Application Publication
(10) Pub. No.: US 2024/0083920 A1
(43) Pub. Date: Mar. 14, 2024

(54) LOW MOLECULAR WEIGHT CORROLE COMPOSITIONS

(71) Applicant: TECHNION RESEARCH & DEVELOPMENT FOUNDATION LTD, Haifa (IL)

(72) Inventors: Zeev GROSS, Haifa (IL); Atif MAHAMMED, Haifa (IL)

(73) Assignee: TECHNION RESEARCH & DEVELOPMENT FOUNDATION LTD, Haifa (IL)

(21) Appl. No.: 18/260,323

(22) PCT Filed: Jan. 11, 2022
(86) PCT No.: PCT/IL2022/050037

§ 371 Date: Jul. 4, 2023

Related U.S. Application Data
(60) Provisional application No. 63/137,152, filed on Jan. 14, 2021.

(51) Int. Cl. C07F 5/00; B01J 31/18; B01J 35/00; C07D 487/22; C07F 13/00; C07F 15/02; C07F 15/06

(52) U.S. Cl. CPC C07F 5/003; B01J 31/1815; B01J 35/004; C07D 487/22; C07F 13/005; C07F 15/025; C07F 15/065; B01J 2231/005; B01J 2531/32; H01M 4/9008

(57) ABSTRACT

Embodiments of the invention relate to a corrole according to formula [I]; wherein R1, R2, and R3 are each independently H, —COOH, CF3, or a halide selected from the group consisting of F—, Cl—, Br— and I, with the proviso that R1, R2, and R3 can not all be CF3, and when the compound is of Formula II, wherein M is a metallic ion or an elemental ion selected from the group consisting of an elemental ion of group 13-16 in row 3 or above and boron, preferably selected from the group consisting of: Fe, Mn, Ga, P, Mo, Re, Co and Cu, or a salt thereof. Methods for treatment, catalysis, and detection using the compounds are also described.

### CROSS REFERENCE TO RELATED APPLICATIONS

Benefit is claimed to U.S. Provisional Patent Application No. 63/137,152, filed Jan. 14, 2021, the contents of which are incorporated by reference herein in their entirety.

### TECHNICAL FIELD

Embodiments of the invention relate to corroles, compositions comprising them, and uses thereof.

### BACKGROUND

Corroles are organic molecules having a contracted porphyrin ring comprising nineteen carbon atoms and 4 nitrogen atoms and are capable of binding transition metals. Various transition metal complexes of corroles were found to have physiological effects in mammals including but not limited to antioxidant or anticancer effects.

### SUMMARY

Embodiments of the invention relate to a corrole according to formula [I];

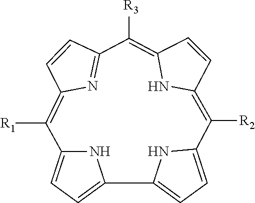

or according to Formula [II]

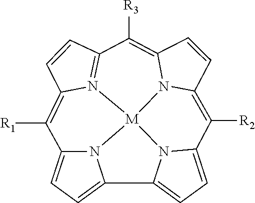

wherein R¹, R², and R³ are each independently H, —COOH, CF₃, or a halide selected from the group consisting of F—, Cl—, Br— and I, with the proviso that R¹, R², and R³ can not all be CF₃, and when the compound is of Formula II, wherein M is a metallic ion or an elemental ion selected from the group consisting of an elemental ion of group 13-16 in row 3 or above and boron, preferably selected from the group consisting of: Fe, Mn, Ga, P, Mo, Re, Co and Cu, or a salt thereof.

The foregoing and other objects, features, and advantages will become more apparent from the following detailed description, which proceeds with reference to the accompanying figures.

### BRIEF DESCRIPTION OF FIGURES

Non-limiting examples of embodiments are described below with reference to figures attached hereto that are listed following this paragraph. Identical structures, elements or parts that appear in more than one figure are generally labeled with a same numeral in all the figures in which they appear, and a numeral labeling an icon representing a given feature in a figure may be used to reference the given feature. Dimensions of components and features shown in the figures are chosen for convenience and clarity of presentation and are not necessarily shown to scale.

FIG. 1 depicts exemplary corroles according to some embodiments, their molecular weight and the log P of selected examples;

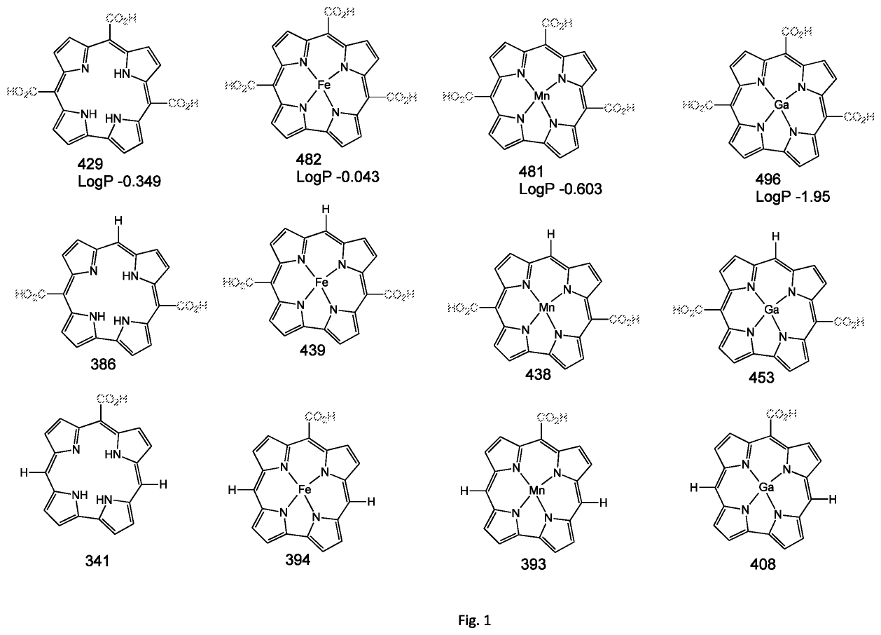

FIG. 2 depicts exemplary metal corrole adsorption to carbon, illustrating their potential for catalysis;

FIG. 3 shows graphs depicting current catalytic performance of metal corroles regarding hydrogen production from aqueous protons, versus the same carbon electrode but without a corrole adsorbed onto it;

FIG. 4 shows a graph depicting UV and visible light absorption of various wavelengths when tested with a metal corrole alone, metal corrole with apomyoglobin and with apomyoglobin alone, indicating binding between the metal corrole and the apomyoglobin;

FIG. 5 shows a graph depicting UV and visible light absorption of various wavelengths when tested using a metal corrole adsorbed onto tin oxide semiconducting substrate versus the metal corrole in solution;

FIG. 6 shows UV-Vis spectra of thin film of iron corrole attached on glass plate under argon (orange line, designated “Ar”) and under nitric oxide (blue line, designated “NO”);

FIG. 7 shows cyclic voltammograms of hydrazine oxidation using Vulcan XC72R carbon electrodes either without or with three types of cobalt-containing corroles; and

FIG. 8A shows cyclic voltammograms of hydrogen production; FIG. 8B shows a charge curve of (cor)Mo(O) adsorbed onto Vulcan XC72R carbon during 15 hours of bulk electrolysis at applied potential of −0.8V; and FIG. 8C shows hydrogen generation in the form of bubbles on the surface of glassy carbon electrode modified by (cor)MO(O) after 10 minutes of bulk electrolysis at applied potential of −0.8V.

### DETAILED DESCRIPTION

### Terms and Definitions

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

### Overview of Several Embodiments

### Novel Compounds

According to one embodiment, provided is a corrole according to formula [I];

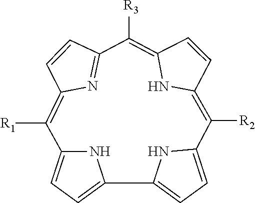

or according to Formula [II]

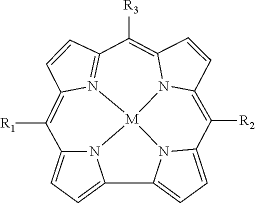

wherein R¹, R², and R³ are each independently H, —COOH, CF₃, or a halide selected from the group consisting of F—, Cl—, Br— and I, with the proviso that R¹, R², and R³ can not all be CF₃, and when the compound is of Formula II, wherein M is a metallic ion or an elemental ion selected from the group consisting of an elemental ion of group 13-16 in row 3 or above and boron, preferably selected from the group consisting of: Fe, Mn, Ga, P, Mo, Re, Co and Cu, or a salt thereof, preferably a pharmaceutically acceptable salt thereof. A “salt” is a salt of corrole which has been modified by making acid or base salts of the compounds. A “pharmaceutically acceptable salt” refers to the relatively non-toxic, inorganic and organic acid or base addition salts of compounds of the present invention.

A corrole described herein may be present in the form of a complex wherein M is either unsubstituted in the axial positions, or is substituted by one or two axial ligands. These ligands may be optionally bound or complexed to the atom M. When M is in the +3 oxidation state, one or both of the ligands may be neutral molecules such as H₂O, NH₃, trialkylamines, aromatic amines, trialkyl- or triaryl-phosphines, or solvent molecules. When M is in the +4 oxidation states, one or both of the ligands may be anionic ligands such as halides, hydroxide and similar. When M is in the +5 oxidation state, one axial ligand may be charged oxygen (O²⁻), nitrogen (N₃₋) or F⁻ or ⁻OH.

### Medicaments, Pharmaceutical Compositions, or Treatments

Previously described corroles have shown to have activity in various models, including atherosclerosis models, as described in PCT Application Publication WO 2009/027965, incorporated herein by reference, and cell protective effects, as described in US Patent Application Publication 2014/0045809, incorporated herein by reference, and diabetes models in U.S. Pat. No. 9,572,816, incorporated herein by reference.

Novel corroles described herein are surprisingly advantageous in that they have molecular weight of less than 500, thereby being suitable for administration to human subjects for treatment of a variety of diseases. Similarly, novel corroles described herein are surprisingly advantageous in that they have relatively high water-solubility relative to corroles previously described, thereby being suitable for administration to human subjects for treatment of a variety of diseases. In addition, they have shown effect as catalysts for various reactions, including photocatalysis.

According to an embodiment the corrole is suitable for administration to human subjects as it has a log P of less than 5. P is an expression of the octanol/water partition coefficient of concentration of a substance in the octanol-rich phase divided by the concentration of the substance in the aqueous phase at room temperature, expressed in logarithmic form. A higher log P indicates lipophilicity, and a lower log P indicates more hydrophilicity. A log P of 0 indicates equal solubility in water and in octanol.

According to an embodiment, provided is a pharmaceutical composition comprising a compound according to formula [I];

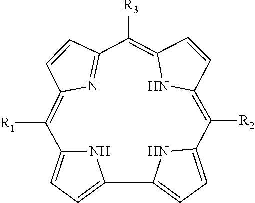

or according to Formula [II]

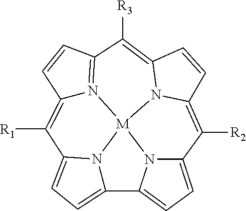

wherein, R¹, R², and R³ are all COOH or wherein at least one of them is COOH and the other(s) are either H or —CF₃, and when the compound is of Formula II, wherein M is a metallic ion or an elemental ion selected from the group consisting of an elemental ion of group 13-16 in row 3 or above and boron, preferably selected from the group consisting of Fe, Mn, P and Ga. Additionally, the compound or composition comprising it may be for use in treatment of disease.

In some embodiments, a pharmaceutical composition contains a pharmaceutically acceptable carrier. As used herein the term “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some embodiments, a pharmaceutical composition is formulated for delivery via any route of administration. As used herein the term “route of administration” refers to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

In some embodiments, the present invention is directed to a composition for use in photodynamic therapy. In some embodiments, the present invention is directed to a composition for use in asymmetric catalysis. In some embodiments, the present invention is directed to a composition for use in Dye Sensitized Solar Cells. In some embodiments, the present invention is directed to a composition for use in in optical imaging of cells. In some embodiments, the present invention is directed to a composition for use in sonodynamic therapy.

### Catalysts

According to one aspect, provided is a corrole according to formula [I];

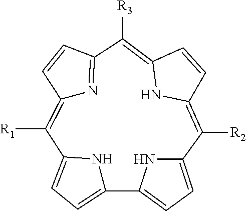

or according to Formula [II]

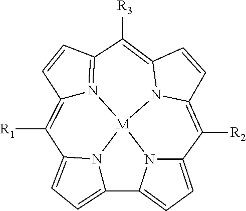

wherein R¹, R², and R³ are each independently H, or CF₃, or COOH with the proviso that R¹, R², and R³ can not all be CF₃, and when the compound is of Formula II, M is selected from the group consisting of Fe, Mn, Ga, P, Mo, Re, Co and Cu, for use as a catalyst.

Corroles described herein, according to an embodiment, may act as electrocatalysts or photo-catalysts for energy-associated applications such as hydrogen production from water. The small size of corroles described herein, preferably those corroles with more hydrogen-substituents as R groups, may be adsorbed to a greater extent to substrates such as porous substrates, comprising metal oxides or conduction metals. Heterogenous catalysts may be formed using such novel corroles and appropriate substrates.

According to an embodiment, the aforementioned corrole is for use as an electrocatalyst. In particular, the corrole may have the structure of formula I or II wherein R¹, R², and R³ are each independently H, or CF₃, with the proviso that R¹, R², and R³ can not all be CF₃, wherein when the compound is of Formula II, M is selected from the group consisting of Fe, Mn, Mo, Re, Co and Cu.

According to an embodiment, the aforementioned corrole is for use as a photocatalyst. In particular, the corrole may have the structure of formula I or II wherein R¹, R², and R³ are each independently H, —COOH, or CF₃, with the proviso that R¹, R², and R³ can not all be CF₃, wherein when the compound is of Formula II, M is selected from the group consisting of P and Ga. Optionally, for use as a catalyst, a corrole described herein may be adsorbed to a substrate, such as a micro- or nano-porous carbon electrode.

### Sensors

According to an embodiment, the aforementioned corrole is for use as a component of a sensor. In particular, the corrole may have the structure of formula I or II wherein R¹, R², and R³ are each independently H, —COOH, or CF₃, with the proviso that R¹, R², and R³ can not all be CF₃, wherein when the compound is of Formula II, M is selected from the group consisting of Fe and Co. Exemplary uses include a sensor for NO gas.

### Photosensitizers

According to an embodiment, the aforementioned corrole is for use as a component of a photosensitizer. Optionally, it may be bound to titanium dioxide semiconductor. In particular, the corrole may have the structure of formula I or II wherein R¹, R², and R³ are all COOH or wherein at least one of them is COOH and the other(s) are either H or —CF₃, wherein when the compound is of Formula II, M is selected from the group consisting of P and Ga. Exemplary uses include a photosensitizer for a photovoltaic cell. Optionally, the corrole may be complexed to a substrate such as titanium oxide.

### Methods for Treatment

According to an aspect of some embodiments of the present invention there is provided a method for treating a disease in a subject in need thereof. In some embodiments the method comprises administering a therapeutically effective amount of a composition according to the present invention to a subject, thereby treating a disease.

In some embodiments, there is provided a method for treating a disease in a subject in need thereof, comprising administering a therapeutically effective amount of a composition comprising a corrole according to formula [I], or formula [II] wherein, wherein R¹, R², and R³ are each independently —COOH or wherein at least one of them is COOH and the other(s) are either H or —CF₃, and when the compound is of Formula II, M is selected from the group consisting of Fe, Mn, P and Ga.

Preferably, amounts administered for treating a disease may range from 0.1 mg/kg per

day, to 200 mg/kg per day.

### Method of Treating Cancer

In some embodiments, there is provided a method for treating cancer in a subject in need thereof, comprising administering a therapeutically effective amount of a composition according to the present invention to a subject, thereby treating cancer.

In some embodiments, there is provided a method for treating a disease associated with oxidative/nitrosative stress (excessive amounts of reactive oxygen/nitrogen species.)

In some embodiments, there is provided a method for photodynamic therapy (PDT). In some embodiments, there is provided a method for sonodynamic therapy (SDT). In some embodiments, there is provided a method for photodynamic therapy (PDT) and sonodynamic therapy (SDT). In some embodiments, there is provided a method for photodynamic therapy (PDT) comprising the steps of administering a therapeutically effective amount of a composition according to the present invention to a subject, and irradiating the subject with energy source at a wavelength capable of exciting the composition. In some embodiments, a wavelength capable of exciting the composition is capable of achieving tissue-penetration.

In some embodiments, there is provided a method for sonodynamic therapy (SDT), comprising the steps of administering a therapeutically effective amount of a composition according to the present invention to a subject, and irradiating the subject with energy source at a wavelength capable of exciting the composition.

In some embodiments, there is provided a method for photodynamic therapy (PDT) and sonodynamic therapy (SDT), comprising the steps of administering a therapeutically effective amount of a composition according to the present invention to a subject, and irradiating the subject with energy source at a wavelength capable of exciting the composition.

In some embodiments, the energy source is selected from light, ultrasound, magnetic force, electromagnetic radiation, LEDs or lasers in the UV-vis electromagnetic spectrum or near infrared.

In some embodiments, the wavelength is between 350 nm to about 900 nm. In some embodiments, the wavelength is between 400 nm to about 900 nm, 450 nm to about 900 nm, 500 nm to about 900 nm, 550 nm to about 900 nm, 600 nm to about 900 nm, or 650 nm to about 900 nm, including any range therebetween.

In some embodiments, irradiating is a plurality of times. In some embodiments, a plurality of times is to achieve treatment effect.

As used herein, the term “sonodynamic therapy” refers to a method involving the combination of ultrasound and a sonosensitiser in which activation of the sonosensitiser by acoustic energy results in the generation of reactive oxygen species, such as singlet oxygen.

As used herein, the term “photodynamic therapy” refers to a process whereby light of a specific wavelength is directed to tissues undergoing treatment or investigation that have been rendered photosensitive through the administration of a photoreactive or photosensitizing agent. The objective may be either diagnostic, where the wavelength of light is selected to cause the photoreactive agent to fluoresce, thus yielding information about the tissue without damaging the tissue, or therapeutic, where the wavelength of light delivered to the target tissue under treatment causes the photoreactive agent to undergo a photochemical interaction with oxygen in the tissue under treatment that yields high energy species, such as singlet oxygen, causing local tissue lysing or destruction.

As used herein, the term “irradiating” and “irradiation” refers to exposing a subject to all wavelengths of light. In some embodiments, the irradiating wavelength is selected to match the wavelength(s) which excite the photosensitive compound. In some embodiments, the radiation wavelength matches the excitation wavelength of the photosensitive compound and has low absorption by the non-target tissues of the subject, including blood proteins.

Irradiation is further defined herein by its coherence (laser) or non-coherence (non-laser), as well as intensity, duration, and timing with respect to dosing using the photosensitizing compound. The intensity or fluence rate must be sufficient for the light to reach the target tissue. The duration or total fluence dose must be sufficient to photoactivate enough photosensitizing compound to act on the target tissue.

According to an aspect of some embodiments of the present invention there is provided a method for imaging a condition in a subject. In some embodiments, the method comprises administering an effective amount of a composition according to the present invention to the subject, wherein M is an imaging agent and applying a magnetic field, X-radiation, UV-vis light or any combination thereof to the subject, thereby imaging the condition in the subject.

In some embodiments, imaging is MRI imaging, fluorescence imaging, positron-emission tomography (PET), or any combination thereof. According to an embodiment, the method of imaging uses a compound in which M=Mn or M=Fe.

According to an aspect of some embodiments of the present invention there is provided a method for imaging a condition in a subject and diagnosing a disease in a subject. In some embodiments, the method comprises, administering an effective amount of a composition according to the present invention to the subject, wherein M is an imaging agent and applying a magnetic field, X-radiation, UV-vis light or any combination thereof to the subject, quantifying a signal intensity of an image of the subject, and comparing the signal intensity with a reference, wherein said signal intensity is greater than the reference is indicative of the subject being afflicted with a disease, thereby diagnosing a disease in the subject.

In some embodiments, a reference is a predetermined signal intensity indicative of a healthy subject.

In some embodiments, compositions described herein are radiophores and emit radiation that is useful in diagnostic and/or monitoring methods employing positron emission tomography. The compounds emit positron radiation capable of producing a pair of annihilation photons moving in opposite directions, the annihilation photons are produced as a result of positron annihilation with an electron. In some embodiments, the radiophore is a radioisotope linked to another chemical structure.

In some embodiments, the radiophore includes a positron-emitting isotope having a suitable half-life and toxicity profile. In some embodiments, the radioisotope has a half-life of more than 30 minutes, more than 70 minutes, more than 80 minutes, more than 90 minutes, more than 100 minutes. In some embodiments, the radioisotope has a half-life of about 30 minutes to about 4 hours, about 70 minutes to about 4 hours, about 80 minutes to about 4 hours, about 90 minutes to about 4 hours, about 100 minutes to about 4 hours, about 30 minutes to about 6 hours, about 70 minutes to about 6 hours, about 80 minutes to about 6 hours, about 90 minutes to about 6 hours, about 100 minutes to about 6 hours, about 30 minutes to about 8 hours, about 70 minutes to about 8 hours, about 80 minutes to about 8 hours, about 90 minutes to about 8 hours, or about 100 minutes to about 8 hours, including any range therebetween.

In some embodiments, a composition according to the present invention comprises a useful positron emitting isotope. In some embodiments, a suitable radiophore is prepared using the fluorine isotope 18F. Other useful positron-emitting isotopes may also be employed, such as 34Cl, 45Ti, 51Mn, 61Cu, 63Zn, 82Rb, 68Ga, 66Ga, 11C, 13N, 15O, and 18F. In one illustrative embodiment, the radioisotope is selected from 64Cu, 68Ga, 66Ga, and 18F. Factors that may be included during selection of a suitable isotope include sufficient half-life of the positron-emitting isotope to permit preparation of a diagnostic composition in a pharmaceutically acceptable carrier prior to administration to the patient, and sufficient remaining half-life to yield sufficient activity to permit extra-corporeal measurement by a PET scan. Further, a suitable isotope should have a sufficiently short half-life to limit patient exposure to unnecessary radiation. In an illustrative embodiment, 18F, having a half-life of 110 minutes, provides adequate time for preparation of the diagnostic composition, as well as an acceptable deterioration rate. Further, on decay 18F is converted to 18O.

According to an embodiment, described herein is a corrole according to formula [I];

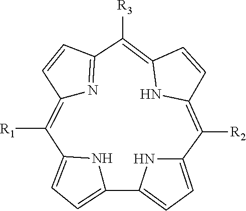

or according to Formula [II]

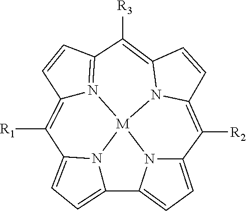

wherein R¹, R², and R³ are each independently H, —COOH, or CF₃ or a halide selected from the group consisting of F—, Cl—, Br— and I, with the proviso that R¹, R², and R³ can not all be CF₃, and when the compound is of Formula II, M is a metallic ion or an elemental ion selected from the group consisting of an elemental ion of group 13-16 in row 3 or above and boron, or a salt thereof. Optionally, M is selected from the group consisting of: Fe, Mn, Ga, P, Mo, Re, Co and Cu, or a salt thereof. Optionally, R¹, R², and R³ are each independently H, —COOH, or CF₃. Optionally, R¹, R² and R³ are each COOH. Optionally, R¹ and R³ are each COOH or CF₃ and R² is H. Optionally, R¹, R² and R³ are each H.

Further described herein is a method for treatment of disease comprising administration to a patient in need thereof a compound according to formula [I] or [II] wherein R¹, R², and R³ are all —COOH, or wherein at least one of them is COOH and the other(s) are either H or —CF₃, and when the compound is of Formula II, M is selected from the group consisting of Fe, Mn, P and Ga. Optionally, the patient suffers from a disease selected from the group consisting of: atherosclerosis, diabetes, a neurodegenerative disease, a disease associated with a oxidative/nitrosative stress and cancer.

Further described herein is a method for catalysis of a reaction comprising introducing a compound according to formula [I] or [II], wherein R¹, R², and R³ are each independently H, or CF₃ or COOH with the proviso that R¹, R², and R³ can not all be CF₃, and when the compound is of Formula II, M is selected from the group consisting of Fe, Mn, Ga, P, Mo, Re, Co and Cu. Optionally, R¹, R², and R³ are each independently H, or CF₃, with the proviso that R¹, R², and R³ can not all be CF₃, wherein when the compound is of Formula II, M is selected from the group consisting of Fe, Mn, Mo, Re, Co and Cu, and wherein the compound acts as an electrocatalyst. Optionally, R¹, R², and R³ are each independently H, —COOH, or CF₃, with the proviso that R¹, R², and R³ can not all be CF₃, wherein when the compound is of Formula II, M is selected from the group consisting of P and Ga, and wherein the compound acts as a photocatalyst. Optionally, the compound is adsorbed to a solid electrode or semiconducting material.

Further described herein is a method for detecting the presence of a chemical agent comprising contacting a compound according to formula [I] or [II] with an chemical agent, and measuring the absorbance of the compound, wherein the compound is of formula I or II wherein R¹, R², and R³ are each independently H, —COOH, or CF₃, with the proviso that R¹, R², and R³ can not all be CF₃, wherein when the compound is of Formula II, M is selected from the group consisting of Fe and Co.

Further described herein is a photosensitizer comprising a corrole according to formula [I] or [II] complexed to a semiconducting metal oxide substrate and wherein the corrole has a structure of formula I or II wherein R¹, R², and R³ are all COOH or wherein at least one of them is COOH and the other(s) are either H or —CF₃, wherein when the compound is of Formula II, M is selected from the group consisting of P and Ga.

Further described herein is a method for imaging a subject comprising administering to the subject an amount of a corrole wherein the corrole has a structure of formula II and wherein M is Mn or Fe, and applying to the subject a magnetic field, X-radiation, UV-vis light.

The following examples are provided to illustrate certain particular features and/or embodiments. These examples should not be construed to limit the disclosure to the particular features or embodiments described.

### EXAMPLES

Reference is now made to the following examples, which together with the above descriptions illustrate some embodiments of the invention in a non-limiting fashion.

### Example 1: Attempt to Synthesize Carboxylic Acid-Substituted Corroles, via Hydrolysis of TEC(Ga)

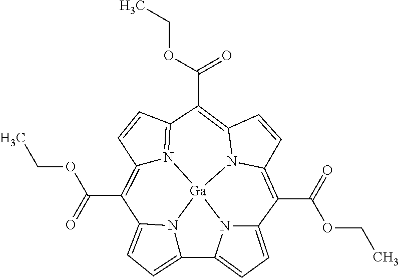

TEC (Ga), a tri-substituted ethyl-ester corrole whose structure is depicted above, was used as a potential starting material. Attempt to hydrolyze the ester with aqueous NaOH were unsuccessful. See G. Canard, D. Gao, A. D'Aléo, M. Giorgi, F.-X. Dang, T. S. Balaban, Chem. Eur. J. 2015, 21, 7760-7771.

### Example 2A: Synthesis of Carboxylic Acid-Substituted Corrole H₃(tcc) from Tris-CF₃ Substituted Corrole H₃(tfc), General Procedure

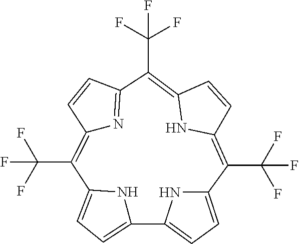 [SMILES: FC(F)(F)c1c2nc(c(C(F)(F)F)c3ccc([nH]3)c3ccc([nH]3)c(C(F)(F)F)c3ccc1[nH]3)C=C2]

Tris-CF₃ substituted corrole H₃(tfc) is depicted above, and can be synthesized using the commercially available precursors, pyrrole and halothane, as described in P. Yadav, S. Khoury, A. Mahammed, M. Morales, S. C. Virgil, H. B. Gray, Z. Gross, Org. Lett. 2020, 22, 3119-3122; and Q.-C. Chen, M. Soll, A. Mizrahi, I. Saltsman, N. Fridman, M. Saphier, Z. Gross, Angew. Chem. Int. Ed. 2018, 57, 1006-1010.

First, H₃(tfc) was treated with basic water and heated for a few hours. This resulted in full consumption of the starting material and quantitative formation of Na₃[H₃(tcc)], which in contrast to H₃(tfc) is freely soluble in water but not in organic solvents. This salt could be extracted into organic solvents via acidification, allowing for isolation of the free-base corrole H₃[H₃(tcc)].

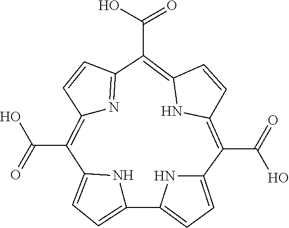 [SMILES: O=C(O)c1c2nc(c(C(=O)O)c3ccc([nH]3)c3ccc([nH]3)c(C(=O)O)c3ccc1[nH]3)C=C2]

Structural assignment of Na₃[H₃(tcc)] and H₃[H₃(tcc)] were based on perfectly matching high-resolution mass spectral data, the absence of ¹⁹F NMR resonances, corrole-characteristic ¹H NMR resonances and J-coupling constants (4-4.5 Hz) of the four β-prrole-CH's, and ¹³C NMR data that clearly disclosed carbonyl resonances at 182 and 180 ppm in the 2:1 ratio that reflects the C₂ axis present in the molecules. The stability of H₃[H₃(tcc)] was best in polar organic solvents like DMSO and acetonitrile; and its sodium salt Na₃[H₃(tcc)] could be stored in aqueous solutions for many weeks.

### Example 2B: Detailed Description of Synthesis of Carboxylic Acid-Substituted Corrole H₃(tcc)

15 mg (30 μmol) of H₃(tfc) was added to 20 mL of aqueous solution of NaOH (20 mM). The suspension was stirred and heated to reflux until all the starting corrole was totally consumed and the hydrolyzed product was totally dissolved. After 4 hr. of reflux the water was evaporated under reduced pressure and the solid residue was dissolved in 1 mL of water and kept in the freezer. In order to characterize this product by NMR and to check purity and yield, 300 μL of the above solution was evaporated to dryness under reduced pressure and re-dissolved in D₂O. The ¹H NMR spectra was indicative of total conversion of the starting corrole to the product (Na₃[H₃(tcc)] in quantitative yields. The acid form of Na₃[H₃(tcc)], 5,10,15-tris(carboxyl)corrole H₃[H₃(tcc)] was obtained by acidifying the aqueous solution of Na₃[H₃(tcc)] with aqueous HCl (1 M) and extraction with methyl ethyl ketone. The organic layer was evaporated under vacuum and re-dissolved in either DMSO-d₆ or CD₃CN for NMR analysis.

Na₃[H₃(tcc)]: ¹H NMR (400 MHz, D₂O, 25° C.): δ=9.45 (d, ³J (H,H)=4.5 Hz, 2H, β-H), 9.28 (d, ³J (H,H)=4.0 Hz, 2H, β-H), 9.23 (d, ³J (H,H)=4.0 Hz, 2H, β-H), 9.19 (d, ³J (H,H)=4.5 Hz, 2H, β-H) ppm. ¹³C NMR (100 MHz, D₂O, 25° C.): δ=181.56, 179.67, 139.58, 138.26, 135.36, 132.42, 126.52, 124.54, 123.15, 116.04, 110.95, 109.21 ppm. UV/Vis (phosphate buffer, pH 7.4): λₘₐₓ (ε, M⁻¹cm⁻¹): 405 (47,100), 567 (8,000), 604 (5,600), 634 (3,100) nm.

H₃[H₃(tcc)]: ¹H NMR (400 MHz, CD₃CN, 25° C.): δ=9.68 (d, ³J (H,H)=4.9 Hz, 2H, β-H), 9.35 (d, ³J (H,H)=4.4 Hz, 2H, β-H), 9.34 (d, ³J (H,H)=4.9 Hz, 2H, β-H), 9.30 (d, ³J (H,H)=4.5 Hz, 2H, β-H), 12.45 (brs, 2H, CO₂H), 11.80 (brs, 1H, CO₂H) ppm. UV/Vis (DMSO): λₘₐₓ (ε, M⁻¹cm⁻¹): 426 (31,500), 583 (3,400), 638 (2,400) nm. HRMS-ESI positive ([M+H]⁺): 431.0977; calculated for C₂₂H₁₅N₄O₆⁺: 431.0986. 1-octanol/water log P=−0.349.

### Example 3A: General Procedure for Preparation of Carboxylic Acid-Substituted Metal Corrole M(tcc) from Tris-CF₃ Substituted Metal Corrole M(tfc)

Various transition and post-transition elemental ion chelated by corroles, were prepared using the following: Mn, Fe, Ga and P in the form of PF₂ or P(OH)₂. As starting materials, H₃(tfc) was prepared as in Example 2. Metal or non-metal substituted corroles were prepared from the following starting material:

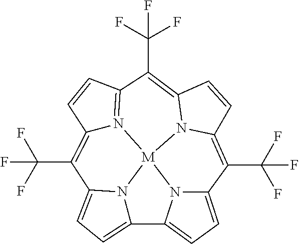

when M represented Mn, Fe, Ga and P(OH)₂, called (tfc)Mn, (tfc)Fe, (tfc)Ga and (tfc)P(OH)₂ respectively. Hydrolysis of the CF₃ groups converted them to sodium salts of the carboxylic acid substituted corroles, designated Na₃[(tcc)Mn], Na₃[(tcc)Fe], Na₃[(tcc)Ga], and Na₃[(tcc)P(OH)₂], respectively. All these complexes were very stable, even when transferred into organic solvents via acidification of the basic aqueous solutions. This allowed for the safe determination of their log P values: −0.60, −0.04, −1.95 and −2.31 for (tcc)Mn, (tcc)Fe, (tcc)Ga, and (tcc)P(OH)₂, respectively. These compounds had the following structure:

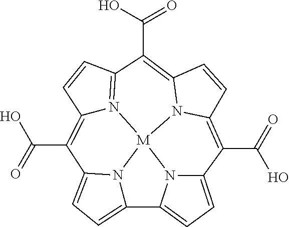

when M represented Mn, Fe, Ga and P(OH)₂. These log P values, of lower than 5 indicate suitability for development as pharmaceutical candidates.

### Example 3B: Detailed Description of Synthesis of Tris-CF₃ Substituted Metal Corrole M(tfc)

### 5,10,15-tris(trifluoromethyl)corrolato cobalt(III) (bis)pyridine, (tfc)Co(py)₂

A solution of H₃(tfc) (25 mg, 50 μmol) and Co(OAc)₂·4H₂O (30 mg, 120 mmol) in pyridine (10 mL) was heated at 100° C. for 15 min. The solvent was evaporated, and the green residue was passed through a column of silica with CH₂Cl₂/hexane/pyridine (1:4:0.01) as eluent. After recrystallization from CH₂Cl₂/hexane/pyridine (1:1:0.01), (tfc)Co(py)₂ was obtained in 80% yield. Single crystals were grown by slow evaporation of CH₂Cl₂/hexane/pyridine solvent system. ¹H NMR (400 MHz, C₆D₆, 25° C.) δ 9.98 (m, J=4.9, 2.2 Hz, 4H), 9.73 (dq, J=4.5, 2.8 Hz, 2H), 9.32 (d, J=4.5 Hz, 2H), 4.37 (t, J=7.5 Hz, 2H), 3.58 (t, 7.0 Hz, 4H), 0.46 (d, J=5.7 Hz, 4H) ppm. ¹³C NMR (100 MHz, C₆D₆, 25° C.): δ=144.99, 142.21, 138.67, 137.91, 134.55, 131.87, 131.46, 131.34, 129.82 (q, ³J_{CF}=5.9 Hz), 129.56, 128.58 (q, ³J_{CF}=5.5 Hz), 121.58, 120.84, 101.53 (q, ²J_{CF}=30.7 Hz), 98.78 (q, ²J_{CF}=32.7 Hz) ppm. ¹⁹F NMR (377 MHz, C₆D₆) δ −36.77 (s, 3F), −39.88 (s, 6F) ppm. UV/Vis (CH₂Cl₂): λₘₐₓ (ε, M⁻¹cm⁻¹): 436 (91,000), 511 (8,000), 548 (12,500), 587 (32,000) nm. HRMS (ACPI, positive mode) for C₂₂H₈F₉N₄Co (M⁺): m/z=557.9937 (calculated), 557.9971 (observed).

### 5,10,15-tris(trifluoromethyl)corrolato iron(III) (bis)pyridine, (tfc)Fe(py)₂

A solution of H₃(tfc) (20 mg, 40 μmol) and FeCl₂·2H₂O (40 mg, 0.25 mmol) in pyridine (10 mL) was heated until reflux for 15 min under nitrogen. The solvent was evaporated, and the residue was passed through a column of silica with ether/pyridine (1:0.01) as eluent. (tfc)Fe(py)₂ was obtained in 83% yield (23.6 mg, 33 μmol). X-ray quality single crystals were grown in diethylether/hexane/pyridine solvent system. ¹H NMR (400 MHz, C₆D₆, 25° C.) δ −105.40, −54.71, −24.67, 5.20, 8.98 (pyr), 10.97 (pyr) ppm. ¹⁹F NMR (377 MHz, C₆D₆) δ −0.78 (br s, 3F), −8.29 (br s, 6F) ppm. UV/Vis (benzene): λₘₐₓ (ε, M⁻¹cm⁻¹): 398 (24,000), 548 (6,000), 683 (2,000) nm. HRMS (ACPI, positive mode) for C₂₂H₈F₉N₄Fe (M⁺): m/z=554.9949 (calculated), 554.9997 (observed).

### 5,10,15-tris(trifluoromethyl)corrolato rhenium(V)(oxo), (tfc)Re(O)

A solution of H₃(tfc) (25 mg, 50 μmol) and excess of Re₂(CO)₁₀ (100 mg, 0.15 mmol) in decalin (5 mL) was heated to 160° C. under an argon atmosphere for 1 hr. The reaction mixture was directly loaded over silica gel/Hexane. The decalin solvent was eluted with n-hexane and then 5% DCM in n-hexane was used to elute red colored (tfc)Re(O) containing fraction. The solvent was evaporated, and the residue was recrystallized in n-hexane. (tfc)Re(O) was obtained in 31% yield (11.0 mg, 15.7 μmol). Red colored X-ray quality crystals were obtained by slow evaporation of CH₂Cl₂/hexane at room temperature. ¹H NMR (400 MHz, C₆D₆, 25° C.) δ 9.74 (m, 4H), 9.71 (dq, J=4.9, 2.3 Hz, 2H), 9.09 (d, J=4.7 Hz, 2H) ppm. ¹³C NMR (100 MHz, C₆D₆, 25° C.): δ=143.24, 141.43, 139.72, 133.90, 128.03, 127.89, 126.94 (q, ³J_{CF}=4.4 Hz), 125.53 (q, ³J_{CF}=7.3 Hz), 118.56, 106.30 (q, ²J_{CF}=32.0 Hz) ppm. ¹⁹F NMR (377 MHz, C₆D₆) δ −40.12 (s, 3F), −42.84 (s, 6F) ppm. UV/Vis (toluene): λₘₐₓ (ε, M⁻¹cm⁻¹): 433 (28,000), 553 (3,000), 593 (5,000). HRMS (ACPI, positive mode) for C₂₂H₈F₉N₄ReO (M⁺): m/z=702.0111 (calculated), 702.0166 (observed).

### Example 3C: Detailed Description of Synthesis of Carboxylic Acid-Substituted Metal Corrole M(tcc)

Na₃[(tcc)P(OH)₂], Na₃[(tcc)Ga], Na₃[(tcc)Fe], Na₃[(tcc)Mn], were prepared by the same procedure: addition of (tfc)P(F)₂, (tfc)Ga, (tfc)Fe, (tfc)Mn, into 20 mM NaOH aqueous solutions and heating to reflux until all the starting compounds were consumed and dissolved in water. Acidification of the aqueous solutions containing the carboxylate salts of the water soluble corroles was used for transferring the neutral corroles into organic solvents.

Na₃[(tcc)Ga]: ¹H NMR (400 MHz, D₂O, 25° C.): δ=9.39 (d, ³J (H,H)=4.6 Hz, 2H, β-H), 9.16 (d, ³J (H,H)=4.6 Hz, 2H, β-H), 9.13 (brs, 4H, β-H) ppm. ¹³C NMR (100 MHz, D₂O, 25° C.): δ=180.15, 166.02, 142.36, 139.27, 135.14, 135.02, 127.88, 124.00, 115.12, 113.66 ppm. UV/Vis (phosphate buffer, pH 7.4): λₘₐₓ (ε, M⁻¹cm⁻¹): 416 (78,800), 573 (8,700), 602 (11,100) nm.

H₃[(tcc)Ga]: ¹H NMR (400 MHz, DMSO-d₆, 25° C.): δ=9.92 (d, ³J (H,H)=4.6 Hz, 2H, β-H), 9.58 (d, ³J (H,H)=4.0 Hz, 2H, β-H), 9.44 (d, ³J (H,H)=4.6 Hz, 2H, β-H), 9.38 (d, ³J (H,H)=4.0 Hz, 2H, β-H),13.80 (brs, 3H, CO₂H) ppm. UV/Vis (DMSO): λₘₐₓ (ε, M⁻¹cm⁻¹): 405 (23,800), 430 (93,000), 560 (4,000), 600 (11,100) nm. HRMS-ESI positive ([M+H]⁺): 497.0062; calculated for C₂₂H₁₂GaN₄O₆⁺: 497.0007. 1-octanol/water log P=−1.950.

Na₃[(tcc)Fe]: ¹H NMR (400 MHz, D₂O, 25° C.): δ=−61.2, −53.5, −28.9 ppm. UV/Vis (phosphate buffer, pH 7.4): λₘₐₓ (ε, M⁻¹cm⁻¹): 390 (32,000), 555(5,500), 747 (1,350) nm.

H₃[(tcc)Fe]: ¹H NMR (400 MHz, DMSO-d₆, 25° C.): δ=−101.2, −58.0 ppm. UV/Vis (DMSO): λₘₐₓ (ε, M⁻¹cm⁻¹): 350 (20,500), 417 (30,000), 560 (8,100), 700 (2,700). HRMS-ESI negative ([M−H]⁻): 481.9981; calculated for C₂₂H₁₀FeN₄O₆⁻: 481.9950. 1-octanol/water log P=−0.043.

Na₃[(tcc)Mn]: λₘₐₓ (ε, M⁻¹cm⁻¹): 308 (15,000), 390 (19,400), 419 (20,000), 483 (13,000), 577 (8,300), 632 (7,600).

H₃[(tcc)Mn]: UV/Vis (DMSO): λₘₐₓ (ε, M⁻¹cm⁻¹): 323 (15,500), 420 (25,000), 481 (36,000), 626 (11,300). HRMS-ESI negative ([M−H]⁻): 481.0075; calculated for C₂₂H₁₀MnN₄O₆⁻: 480.9981. 1-octanol/water log P=−0.603.

Na₃[(tcc)P(OH)₂]: ¹H NMR (400 MHz, D₂O, 25° C.): δ=9.39 (dd, ⁴J (P,H)=2.72 Hz, ³J (H,H)=4.8 Hz, 2H, β-H), 9.18 (dd, ⁴J (P,H)=2.32 Hz, ³J (H,H)=6.48 Hz, 6H, β-H) ppm. UV/Vis (phosphate buffer, pH 7.4): λₘₐₓ (ε, M⁻¹cm⁻¹): 388 (44,921), 409 (1,78,575), 562 (12,024), 584 (27,466) nm.

H₃[(tcc)P(OH)₂]: ¹H NMR (400 MHz, DMSO-d₆, 25° C.): (tcc)P(OH)₂:(tcc)P(O), 2:1 ratio; δ=9.98 (dd, ³J (H,H)=5.12 Hz, 2H, β-H), 9.85 (dd, ³J (H,H)=5.12 Hz, 1H, β-H), 9.73 (t, ³J (H,H)=4.6 Hz, 2H, β-H), 9.63-9.66 (unresolved dd, ³J (H,H)=4.4 Hz, ⁴J (P,H)=3.4, 4H, β-H), 9.60 (dd, ³J (H,H)=4.6 Hz, ⁴J (P,H)=3.2 Hz, 1H, β-H), 9.51 (dd, ³J (H,H)=4.9 Hz, ⁴J (P,H)=2.8 Hz, 1H, β-H), 9.48 (dd, ³J (H,H)=4.5 Hz, ⁴J (P,H)=2.2 Hz, 1H, β-H) ppm. UV/Vis (DMSO): λₘₐₓ (ε, M⁻¹cm⁻¹): 399 (34,524), 423 (1,51,210), 548 (6,482), 592 (18,393) nm. HRMS-ESI positive ([M−H]⁻): 491.0387; calculated for C₂₂H₁₂N₄O₈P⁻: 491.0393. 1-octanol/water log P=−2.31.

### Example 4: Formation of Bis-Substituted Carboxylic Acid-Substituted Corrole ABA H₃(dcc) Using Bis-CF₃ Substituted Corrole ABA H₃(dfc), General Description

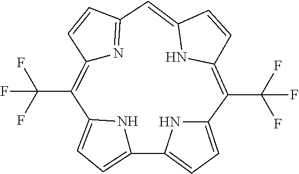 [SMILES: FC(F)(F)c1c2nc(cc3ccc([nH]3)c(C(F)(F)F)c3ccc([nH]3)c3ccc1[nH]3)C=C2]

In the case of bis-substitution, the naming convention used was ABA wherein the substitutions were represented as follows:

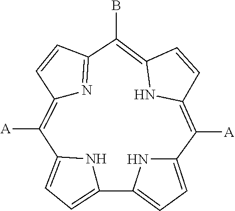

Bis-CF₃ substituted corrole ABA H₃(dfc) was prepared via cyclocondensation of two equivalents of CF₃-substituted dipyrromethene with one equivalent formaldehyde. See J. S. Lindsey, Acc. Chem. Res. 2010, 43, 300-311, and R. Orlowski, D. Gryko, D. T. Gryko, Chem. Rev. 2017, 117, 3102-3137.

In a 100 mL two-necked flask fitted with a rubber septum and a reflux condenser connected to an oil bubbler. Na₂S₂O₄ (85%, 1.0 g, 5 mmol) and NaHCO₃ (3.5 g, 42 mmol) were suspended in an MeCN-H₂O mixture (2:1, 30 mL). The suspension was warmed up to 38° C. in an oil bath and a mixture of pyrrole (4.0 g, 60 mmol) and 1-bromo-1-chloro-2,2,2-trifluoroethane (4.0 g, 20 mmol) was injected with a syringe through the septum and stirred vigorously. After 1.5 h, all the inorganic salts dissolved and stirring was continued for another 2 h. Water (50 mL) was added and reaction mixture was extracted with Et₂O (3×30 mL). The combined extracts were washed with water twice and dried (Na₂SO₄). Solvent was removed on a rotary evaporator and gave 4.0 g of a brown oil. The oil was subjected to column chromatography on silica gel (hexanes-DCM, 8:2) gave colorless, cotton wool like needles. Yield, after recrystallization from hexanes, (2.50 g, 58% relative to CF₃CHClBr).

CF₃-DPM (500 mg) and formaldehyde (100 μL of 35% aqueous solution) were added to a 1:1 MeOH:H₂O mixture (v/v, 200 mL) to which aqueous HCl (6 mL of 36% solution) was added after a few minutes. The reaction mixture was stirred at room temperature for 2 h, followed by extraction with DCM, washing with water and drying over sodium sulfate. That solution was treated with DDQ (0.26 g) for 30 min. The solvent was evaporated over rotary evaporator and passed through a column of silica gel. The first eluted fraction (by a 20/80 CH₂Cl₂/hexane mixture) was 5,15-bis(trifluoromethyl)porphyrin (H₂(dfp)) and the second fraction, purple in color, was collected and determined to be the title compound. X-ray quality single crystals of H₂(dfp) and H₃(dfc) were grown by slow evaporation of CH₂Cl₂/n-hexane and CH₂Cl₂/n-heptane, respectively. H₃(dfc) Yield=2.9%. ¹H NMR (400 MHz, CDCl₃, 25° C.) δ=10.27 (s, 1H, meso-H), 9.60-9.56 (q, J=4.8, 2.4 Hz, 2H, β-H), 9.34 (d, J=4.8 Hz, 2H, β-H), 9.16-9.11 (q, J=4.4, 2.4 Hz, 2H, β-H), 9.07 (d, J=4.4 Hz, 2H, β-H); ¹⁹F NMR (377 MHz, CDCl₃) δ=−44.39 (s, 6F, meso-F). UV/Vis (toluene): λₘₐₓ (ε, M⁻¹cm⁻¹): 396 (30,200), 414 (27,400), 546 (5,400), 567 (2,740), 611 (5,500) nm. HRMS (APCI, negative mode) for C₂₁H₁₁F₆N₄ (M−H⁻): m/z=433.0888 (calculated), 433.0932 (observed).

### Example 5A: General Procedure for Preparation of Carboxylic Acid-Substituted Metal Corrole M(dcc) from Bis-CF₃ Substituted Metal Corrole M(dfc)

Hydrolysis of the ABA corroles with A=CF₃ to the bis-carboxylated corroles wherein A=CO₂⁻, was checked for the Co, Re(O), Fe and P(OH)₂ complexes.

### Example 5B: Detailed Description of Synthesis of Bis-CF₃ Substituted Metal Corrole M(dfc)

### 5,15-Bis(trifluoromethyl)(dihydroxy)phosphorus(V) corrole, (dfc)P(OH)₂

Free base H₃(dfc) (40 mg, 0.09 mmol) was dissolved in pyridine with vigorous stirring, followed by addition of 300 μL PCl₃ (each 100 μL in 10 min interval). The reaction mixture was stirred for another 10 min. After completion of reaction, the reaction mixture was quenched with water and extracted using CH₂Cl₂. The organic layer was evaporated to dryness and chromatographed over silica gel column using CH₂Cl₂/hexane (1:1) mixture to afford pure pink colored fraction containing (dfc)P(OH)₂. Crystallization using CH₂Cl₂/n-heptane mixture yield X-ray quality single crystals. Yield=70%. ¹H NMR (400 MHz, CDCl₃, 25° C.) δ. ¹H NMR (400 MHz, CH₃OD, 25° C.) δ 8.79 (s, 1H, meso-H), 8.15 (dd, J=5.24, 2.48 Hz, 2H, β-H), 8.09 (dd, J=5.08, 2.4 Hz, 4H, β-H), 8.79 (dd, J=5.0, 2.64 Hz, 2H) ppm. UV/Vis (CHCl₃): λₘₐₓ: 380 (16,000), 399 (78,600), 528 (2,000), 567 (12,900) nm.

### 5,15-Bis(trifluoromethyl) corrolato rhenium(V)(oxo), (dfc)Re(O)

H₃(dfc) (40 mg, 0.09 mmol) was dissolved in decalin (5 mL) and heated to 160-170° C. under an argon atmosphere. Re₂(CO)₁₀ (100 mg, 0.15 mmol) was added to the solution and heating was continued for 1 hr. The cooled reaction mixture was directly loaded onto a silica/hexane column, the decalin solvent was eluted by using hexane and 5% DCM/hexane was used to elute the red colored rhenium corrole. The solvent was evaporated, and the residue was recrystallized in n-hexane. The single crystals were grown in benzene solution at low temperature (4° C.). Yield=26%. ¹H NMR (400 MHz, CDCl₃, 25° C.) δ 10.57 (s, 1H, meso-H), 10.06 (q, J=4.4, 2.4 Hz, 2H), 10.01 (q, J=4.8, 2.4 Hz, 2H), 9.83 (d, J=4.8 Hz, 2H), 9.69 (d, J=4.92 Hz, 2H) ppm. ¹⁹F NMR (377 MHz, C₆D₆) δ −43.20 (s, 6F) ppm. UV/Vis (toluene): λₘₐₓ: 428 (77200), 552 (8780), 592 (37800). HRMS (APCI, negative mode) for C₂₁H₉F₆N₄ReO (M⁻): m/z=634.0238 (calculated), 634.0243 (observed).

### 5,15-Bis(trifluoromethyl) corrolato cobalt(III) bis-pyridine, (dfc)Co(py)₂

Experimental procedure is same as given for (tfc)Co(py)₂. Single crystals were obtained by slow evaporation of CH₂Cl₂/hexane/pyridine solvent system. ¹H NMR (400 MHz, C₆D₆, 25° C.) δ 10.08 (dq, J=4.84, 2.52 Hz, 2H), 9.89 (s, 1H, meso-H), 9.84 (dq, J=4.52, 2.76 Hz, 2H), 9.44 (d, J=4.52 Hz, 2H), 9.32 (d, J=4.8 Hz, 2H) ppm. ¹⁹F NMR (377 MHz, C₆D₆) δ −39.29 (s, 6F) ppm. UV/Vis (CH₂Cl₂): λₘₐₓ: 425 (53,787), 543 (8,269), 588 (33,974) nm. HRMS (ACPI, negative mode) for C₂₁H₉F₆N₄Co (M⁻): m/z=490.0063 (calculated), 489.9996 (observed).

### 5,15-bis(trifluoromethyl)corrolato iron(III) bis-pyridine, (dfc)Fe(py)₂

Synthetic procedure is same as (tfc)Fe(py)₂. X-ray quality single crystals were grown in diethylether/hexane/pyridine solvent system. UV/Vis (ether/pyridine, 9.5:0.5 (v/v)): λₘₐₓ (ε, M⁻¹cm⁻¹): 398 (14880), 546 (5519), 746 (1564) nm.

### Example 5C: Detailed Description of Synthesis of Bis-Substituted Carboxylic Acid-Substituted Metal Corrole M(dcc)

Na₂[(dcc)P(OH)₂] and Na₂[(dcc)Fe were prepared by addition of (dfc)P(OH)₂, or (dfc)Fe(py)₂ into 20 mM NaOH aqueous solutions and heating to reflux until all the starting compounds were consumed and dissolved in water. Acidification of the aqueous solutions containing the carboxylate salts of the water soluble corroles was used for transferring the neutral corroles into organic solvents.

Na₂[(dcc)P(OH)₂]: ¹H NMR (400 MHz, D₂O, 25° C.): δ=9.75 (brs, 1H, meso-H), 9.37 (brs, 2H, β-H), 9.18 (brs, 4H, β-H), 9.08 (brs, 2H, β-H) ppm. UV/Vis (phosphate buffer, pH 7.4): λₘₐₓ (ε, M⁻¹cm⁻¹): 383 (24,779), 398 (59,336), 562 (4,859), 577 (9,915) nm.

H₂[(dcc)P(OH)₂: ¹H NMR (400 MHz, DMSO-d₆, 25° C.): δ=10.42 (s, 1H, meso-H), 10.07 (t, ³J (H,H)=4.4 Hz, 2H, β-H), 9.82 (t, ³J (H,H)=4.24 Hz, 2H, β-H), 9.68 (dd, ³J (H,H)=4.12 Hz, ⁴J (P,H)=2.64 Hz, 2H, β-H), 9.56 (dd, ³J (H,H)=4.6 Hz, ⁴J (P,H)=2.92 Hz, 2H, β-H), ppm. UV/Vis (DMSO): λₘₐₓ (ε, M⁻¹cm⁻¹): 393 (20,510), 415 (47,301), 543 (2,077), 591 (10,093) nm. 1-octanol/water log P=−1.89.

Na₂[(dcc)Fe]: UV/Vis (phosphate buffer, pH 7.4): λₘₐₓ (ε, M⁻¹cm⁻¹): 380 (13863), 553 (1214) nm.

H₂[(dcc)Fe]: UV/Vis (DMSO): λₘₐₓ (ε, M⁻¹cm⁻¹): 363 (22817), 548 (3068) nm. 1-octanol/water log P=−0.304.

### Example 6A: Formation of Unsubstituted Free Base Corrole H₃(cor) and Metallocorrole M(cor) by Sublimation

Sublimation was used to obtain the following compounds, designated H₃(cor) and metallocorrole (cor)M respectively:

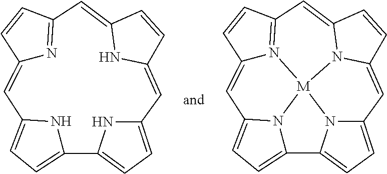

A solid amount of H₃(tcc) or (tcc)M (5 mg) was prepared as above, in examples 2 or 3. M was either Ga, or Fe. The starting material was sublimated under high vacuum at 200° C. The sublimated product was dissolved in benzene for further characterization.

H₃(cor): It appeared that stability was limited due to ambient humidity and light conditions. Without being bound by theory, it appears that electron-withdrawing substituents are required for reducing the π-system electron richness of the corrole macrocycle. Free base unsubstituted corrole was not stable enough to get full characterization details. UV/Vis (C₆H₆): λₘₐₓ (ε, %): 391 (100), 411 (87), 533 (27), 550 (23) 586 (19) nm.

(cor)Ga(py): ¹H NMR (400 MHz, CDCl₃, 25° C.) δ 9.80 (s, 1H, meso-H), 9.59 (s, 1H, meso-H), 9.29 (d, J=4.32 Hz, 2H, β-H), 9.22 (d, J=3.80 Hz, 2H, β-H), 9.17 (d, J=3.84 Hz, 2H, β-H), 9.11 (d, J=4.32 Hz, 2H, β-H) ppm. UV/Vis (C₆H₆): λₘₐₓ (ε, %): 406 (100), 555 (17), 578 (16) nm.

(cor)Fe(py)₂: UV/Vis (C₆H₆): λₘₐₓ (ε, %): 403 (100), 425 (85), 457 (48), 540 (23) nm.

### Example 6B: Additional Routes for Formation of Unsubstituted Metallocorrole M(cor)

Two additional routes were successful in obtaining metallocorroles. The first route involved heating under Argon in decalin, and addition of the metal complexing agent. The second route involved oxidative cyclization of a tetrapyrromethane (bilane) in-situ with metal complexing agent.

### (oxo)Rhenium(V) Corrole, (cor)Re(O)

A solution of H₃[H₃(tcc)] (15 mg, 37.8 μM) in decalin (5 mL) was heated to 160° C. under an argon atmosphere. After 30 minutes of heating H₃[H₃(tcc)] become soluble (initially not soluble in decalin) and form a green solution. Excess of Re₂(CO)₁₀ (100 mg, 0.15 mmol) was added immediately when the decalin solution changed its color from green to pink (after additional heating of 30 minutes) and continued heating for another 40 minutes. The reaction mixture was directly loaded over silica gel/Hexane column. The decalin solvent was eluted with n-hexane and then 20% DCM in n-hexane was used to elute the red colored (embryo)Re(O) containing fraction. The solvent was evaporated, and the residue was recrystallized in n-hexane. (cor)Re(O) was obtained in 18% yield (3.4 mg, 6.8 μM). ¹H NMR (400 MHz, CDCl₃, 25° C.) δ 10.61 (s, 1H, meso-H), 10.41 (s, 1H, meso-H), 9.70 (d, J=4.32 Hz, 2H, β-H), 9.66 (d, J=4.40 Hz, 2H, β-H), 9.62 (d, J=4.60 Hz, 2H, β-H), 9.55 (d, J=4.64 Hz, 2H, β-H) ppm. UV/Vis (CH₂Cl₂): λₘₐₓ (ε, %): 422 (100), 537 (26), 570 (31). HRMS (APCI, positive mode) for C₁₉H₁₁N₄ReO (M⁺): m/z=498.0496 (calculated), 498.0534 (observed).

### (oxo)Molybdenum(V) Corrole, (cor)Mo(O)

A solution of H₃[H₃(tcc)] (15 mg, 37.8 μM) in decalin (5 mL) was heated to 160° C. under an argon atmosphere. After 30 minutes of heating H₃[H₃(tcc)] become soluble (initially not soluble in decalin) and form a green solution. When the decalin solution changed its color from green to pink (after additional heating of 30 minutes) the temperature of the reaction mixture was decreased to 120° C. and excess of Mo(CO)₆ (100 mg, 0.37) were added. The temperature of the solution raised again to 160° C. and heated for another 40 minutes. The reaction mixture was directly loaded over silica gel/Hexane column. The decalin solvent was eluted with n-hexane and then 30% DCM in n-hexane was used to elute the brown colored (embryo)Mo(O) containing fraction. The solvent was evaporated, and the residue was recrystallized in n-hexane. (cor)Mo(O) was obtained in 26% yield (4.0 mg, 9.8 μM). HRMS (APCI, positive mode) for C₁₉H₁₁N₄MoO (M⁺): m/z=408.9992 (calculated), 409.0037 (observed).

### (cor)Co(py)₂

Bilane (90 mg, 0.296 mmol) was dissolved in pyridine (20 ml). Cobalt acetate tetrahydrate (368 mg, 1.477 mmol) was added followed by addition of [Bis(trifluoroacetoxy)iodo]benzene, PIFA (375 mg, 0.872 mmol) at room temperature. Then the temperature was increased to 60° C. and the reaction was stirred for 1 h at 60° C. The solvent was evaporated via rotary evaporator and the crude reaction mixture was passed through a column of silica gel. The first fraction was eluted by DCM/Hexanes/Pyridine solution mixture (1:9:0.01) and was determined to be the titled product. The product was dissolved in 200 ml of hexanes, few drops of pyridine were added and kept in the fridge for overnight followed by filtration to get the product dissolved in hexanes. Solvent was removed via rotary evaporator and the product was recrystallized via pentane/DCM/Pyridine mixture (2:1:0.01) to get purple color crystals in 15% yield (22.0 mg, 43.0 μmol). ¹H NMR (400 MHz, C₆D₆, 25° C.) δ 9.97(s, 1H, meso-H), 9.79 (s, 1H, meso-H), 9.48 (d, J=4.08 Hz, 2H, β-H), 9.49 (d, J=4.40 Hz, 2H, β-H), 9.28 (dd, J=4.28, 1.68 Hz, 4H, β-H), 4.85 (br s, 2H, p-pyr), 4.20 (br s, 4H, rn-pyr), 1.20 (br s, 4H, o-pyr) ppm. HRMS (APCI, positive mode) for C₁₉H₁₁CoN₄ (M⁺): m/z=354.0316 (calculated), 354.0310 (observed).

### (cor)Co(PPh₃)

TPM (200 mg, 0.66 mmol) was dissolved in pyridine (30 ml), Cobalt acetate tetrahydrate (3.3 g, 13.2 mmol) was added followed by addition of PIFA (851 mg, 2 mmol) at room temperature, the color of the solution was reddish-black. Then the temperature was increased to 60° C. Another portion of Cobalt acetate tetrahydrate (1.65 g, 6.6 mmol) was added after 30 min and the reaction was stirred for another 30 min at 60° C. The solvent was evaporated via rotary evaporator and the crude reaction mixture was passed through a column of silica gel. The first fraction was eluted by DCM/Hexanes/Pyridine solution mixture (1:9:0.01 v/v) and triphenylphosphine, (PPh₃, 26 mg, 0.1 mmol) was added directly to this eluted fraction. The solvent was removed via rotary evaporator at 60° C., and to ensure full evaporation of excess of pyridine at 60° C., 4 portions of hexanes were added to the flask that contains the product and removed via rotary evaporator at 60° C. This procedure is important to remove excess of pyridine without the decomposition of the product that sensitive to high temperature required to remove pyridine. Full transformation of (cor)Co(py)₂ to (cor)Co(PPh₃) was determined by TLC. The product was recrystallized from benzene/n-heptane (1:1 v/v) and provided dark red crystals in 8% yield (32.0 mg, 52.0 μmol). X-ray quality single crystals were grown by slow evaporation of a concentrated solution of (cor)Co(PPh₃) in n-heptane/benzene (1:1 v/v) at room temperature. ¹H NMR (400 MHz, C₆D₆) δ 9.62 (s, 1H, meso-H), 9.38 (s, 2H, meso-H), 8.70 (d, J=4.2 Hz, 2H, β-pyrrole-H), 8.47 (d, J=4.4 Hz, 2H, β-pyrrole-H), 8.42 (d, J=4.3 Hz, 2H, β-pyrrole-H), 8.39 (d, J=4.2 Hz, 2H, β-pyrrole-H), 6.63 (m, 3H, para-H of PPh₃), 6.40 (m, 6H, meta-H of PPh₃), 4.73 (m, 6H, ortho-H of PPh₃) ppm. UV/Vis (benzonitrile): λₘₐₓ (ε, M⁻¹cm⁻¹): 368 (40,000), 396 (32,500), 564 (10,700), 536 (8,200) nm. HRMS (APCI, negative mode) for C₁₉H₁₁CoN₄ (M⁻): m/z=354.03158 (calculated), 354.0310 (observed).

Cyclization of the Bilane was done with and without adding oxidizing agent and different mild oxidizing agent were used. In the case of not adding any oxidant, the product was obtained in low yield (3%). Performing the reaction under dioxygen atmosphere gave similar yield. Using PIFA and (Diacetoxyiodo)benzene (3 equivalents in each case) gave a significant increase in the yields in both cases which was the best in the case of using PIFA (15%).

### Example 6C: Additional Routes for Formation of Unsubstituted Metallocorrole (cor)Ga(py)

A solution of H₃(dfc) (20 mg) in 10 mL pyridine was heated and excess amount of GaCl₃ was added. The solution was refluxed for 1 hr. The reaction mixture was evaporated to dryness and dissolved in small amount of DCM containing few drops of pyridine. The solution was loaded over silica gel column already packed with n-hexane containing few mL of pyridine. The bright pink colored (cor)Ga(py) was eluted with 20% DCM in n-hexane and small amount of pyridine. The eluted pink fraction was evaporated, and the residue was recrystallized in benzene.

### Example 7: Preparation of Various Corrole Metal Complexes Adsorbed to Carbon

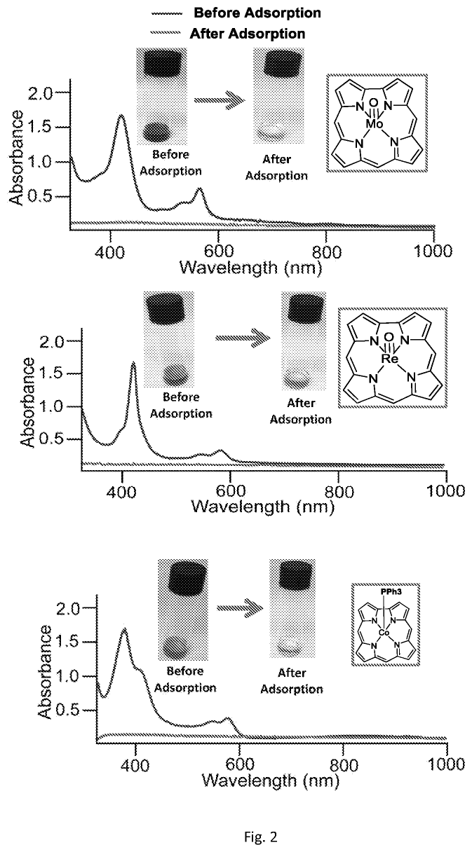

The M(cor) (M=Co, Re, Mo) complexes were prepared as above and adsorbed on Vulcan XC72R carbon by mixing 0.8 mg of corrole and 10 mg of Vulcan XC72R carbon in 1 mL isopropanol. The corresponding solutions were sonicated for 20 min, after which they were stirred overnight at room temperature. The resulting suspensions were centrifuged to separate the carbon nanoparticles from the solution. To calculate the amount of corrole that was adsorbed on the carbon the UV-Vis spectra of the isopropanol solution were measured before and after the adsorption of the corrole on the carbon (FIG. 2). The modified carbon were dried at 45° C. for overnight. The dried carbon was washed with 1 mL isopropanol and again stirred overnight, centrifuged, and dried overnight at 45° C. Examination of the isopropanol solutions before and after the above-described treatment (by both naked eye and UV-vis spectroscopy, FIG. 2) clearly reveals quantitative absorption of the corrole to the carbon. That is in sharp contrast with triarylcorroles, for which only partial binding is obtained by completely identical treatment. In addition, the binding process of the (cor)M complexes occurs very fast and is practically complete with 20-30 minutes.

This example shows the potential for use of metal corrole complexes described herein for catalysis. High loading without heating can provide for an inexpensive, easily obtainable catalyst.

### Example 8: Electrodes and Catalysis Using Corrole Metal Complexes

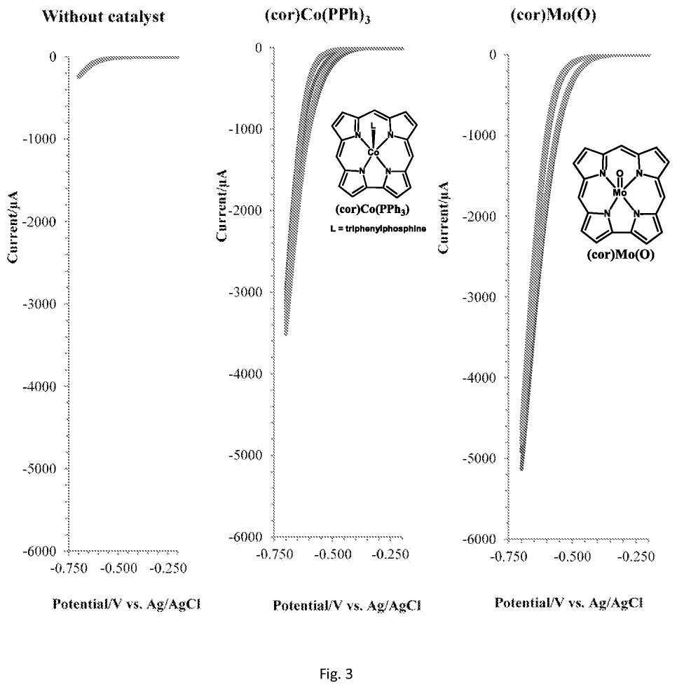

Inks of the corrole-containing carbons were prepared by mixing 1 mg from the solid mixtures described above, with 0.2 mL isopropanol, 0.8 mL H₂O, and 10 μL of Nafion, followed by sonication for 20 min. Small quantities (5 μl) of this kind of inks were dropped on the surface of glassy carbon electrodes (GC), followed by drying the modified electrodes for 40 min at 45° C. This process was repeated once more for each electrode and also for the ink prepared from non-modified Vulcan XC72R. The performance of the electrode prepared from the latter was checked relative to the electrodes modified by (cor)Co(PPh₃) and (cor)Mo(O), by examining the electrocatalytic hydrogen production from protons (FIG. 3).

Electrochemical hydrogen production was performed using 0.5 M H₂SO₄ solution under N₂ by (cor)CoPPh₃ and (cor)Mo(O) catalysts adsorbed onto Vulcan XC72R carbon as the solid support. Ag/AgCl and Pt wire were used as reference and counter electrodes, respectively. The electrode that did not contain the adsorbed molecular catalysts was ineffective down to −0.7 V, while the one with (cor)Co and more so the one with (cor)Mo(O) were very effective according to various parameters: Faradaic yield, early onset potential (low overpotential) and durability (10 cycles with hardly any change).

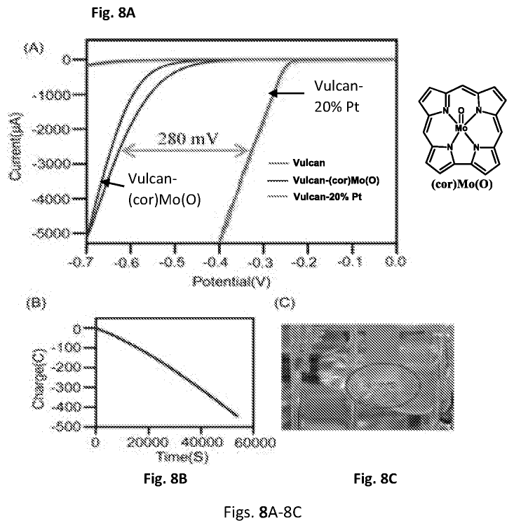

When hydrogen production using (cor)Mo(O) catalyst compared to Vulcan XC72R carbon only, and 20% Pt adsorbed onto Vulcan XC72R carbon the results with (cor)Mo(O) reveal that, even though its electronic effect would suggest it to be a poor catalyst, it is superior due to its very dominant size effect (the dimensions of (cor)Mo(O) is much smaller than other corroles that are substituted by CF₃ or C₆F₅ groups so it is with the smallest size (and hence strongest binding to the Vulcan). The Mo complex catalyzes the transformation of protons to hydrogen gas with 92% Faradaic Efficiency (FE) at only 280 mV overpotential relative to 20% Pt. A cyclic voltammogram showing hydrogen production with these catalysts is shown in FIG. 8.

### Example 9: Electrocatalysis Using (cor)Fe as Compared to 20% Pt

Platinum is a known catalyst, when used in conjunction with a carbon substrate. Minimally substituted iron corrole was adsorbed onto Vulcan carbon xc72 as described above. Electrocatalysis was tested versus 20% Pt on Vulcan xc72 in electrocatalytic reduction of O₂ to H₂O and examined as described in the following references: Angew. Chem. Int. Ed. 2015, 54, 14080-14084. The results are shown in Table 1 below.

TABLE 1

| E vs | | | | | N at peak E |
|---|---|---|---|---|---|
| SHE | Eₒₙₛₑₜ[V] | E_{p/2}[V] | Eₚₑₐₖ[V] | Jₚₑₐₖ[mA/cm²] | (1600 rpm) |
| (cor)Fe | 0.99 | 0.91 | 0.82 | 0.56 | 3.8 |
| 20% Pt | 1.08 | 1.01 | 0.9 | 0.754 | 3.9 |

This example shows that the novel iron corrole described herein is nearly as effective in electrocatalytic reduction as the expensive/rare and hence non-sustainable Platinum-based catalyst. The novel (cor)Fe shows potential as an electrocatalyst and may serve as a catalyst in fuel cells.

Similarly, minimally substituted iron corrole (cor)Fe when adsorbed onto Vulcan xc-72 carbon is more efficient as a catalyst for use in spontaneous decomposition of H₂O₂ to O₂ than regular iron corrole. 0.8 mg iron corrole,10 mg Vulcan carbon and 1 ml isopropanol were mixed together, sonicated for 20 min and stirred overnight at room temperature. The suspension was centrifuged until the carbon-solution was separated. The mother liquor was removed and the carbon was dried over-night at 45° C. This indicates potential use of corroles described herein for applications such as water purification or as additive to fuel cells or batteries where unfortunate formation of H₂O₂ is harmful for the key components such as the proton exchange membrane.

### Example 10: Binding of (tcc)Fe to Apomyoglobin

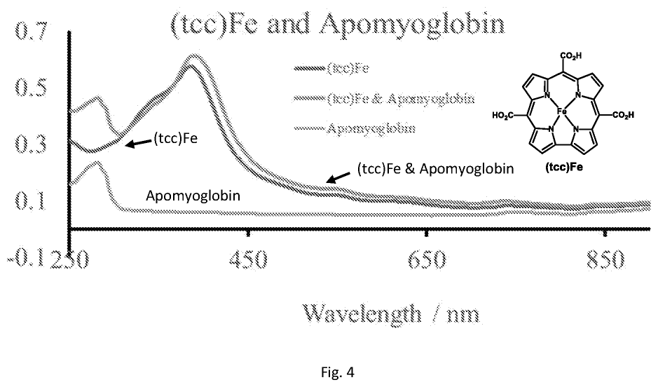

Apomyoglobin was combined with (tcc)Fe by mixing equimolar aqueous solutions of both. According to absorbance spectra of apomyoglobin, apomyoglobin in combination with (tcc)Fe and (tcc)Fe alone, it appears (FIG. 4, best seen by the spectral changes at 300-450 nm) that (tcc)Fe binds apomyoglobin. This shows potential use for (tcc)Fe and potentially other corroles described herein as an agent that has heme-mimicking capability, thereby allowing it to be used as a blood substituent or for enzyme-like activity.

### Example 11: Photocatalysis with Metal Corrole (tcc)Ga

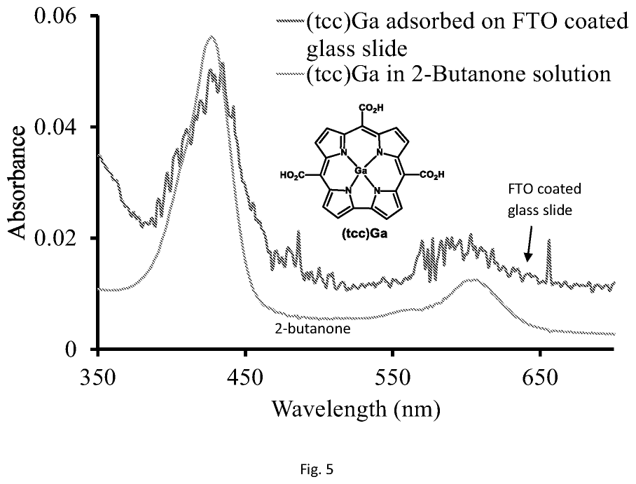

Exemplary corroles described herein such as carboxylated metal corroles may be useful for dye-sensitized solar cells (DSSC). There are many examples for the utility of TiO₂-bound photosensitizer, of which the best known is DSSC (Graetzel type). To confirm that the novel corroles bind semiconducting oxides in an irreversible fashion, the following procedure was applied. Tin oxide coated glass slide was immersed for 18 hours in 2-butanone solution containing (tcc)Ga. The glass slide was then taken out and washed twice with 2-butanone. The UV-Vis spectrum of the glass slide was recorded, and is shown in FIG. 5. FIG. 5 also shows the UV-Vis spectrum of (tcc)Ga in 2-butanone solution. The (tcc)Ga was spontaneously adsorbed to the tin oxide substrate, illustrating its potential use as an agent in photocatalysis.

### Example 12: Use of Corroles as Sensors

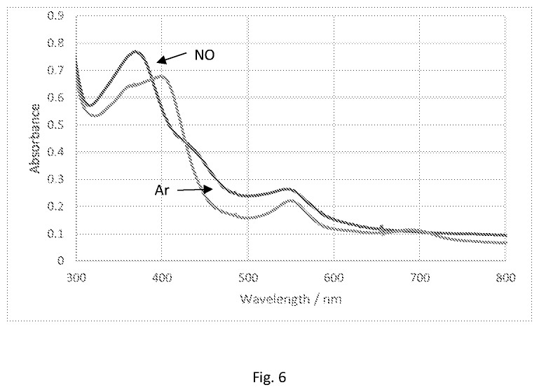

One drop of 2-butanone that contained iron corrole was added on transparent glass plate. After the 2-butanone was evaporated at room temperature, the UV-Vis absorption of the glass was measured before and after treatment with NO gas. The absorbance of UV-Vis absorption differs when in contact with NO gas versus when in an argon environment, as evident by the graph in FIG. 6, indicating the potential use of corroles described herein as sensors for NO.

### Example 13: Hydrazine Oxidation

Hydrazine was catalytically oxidized using electrodes comprising either Vulcan XC72R carbon, or three cobalt complexes adsorbed onto them that differ only in the identity of the 5,10,15-substituents: (tpfc) with three C₆F₅ groups ((tpfc)Co(PPh₃)), (tfc) with three CF₃ groups ((tfc)Co(PPh₃)) and (cor) with no substituents ((cor)Co(PPh₃)). The preparation of (cor)Co(PPh₃), (tpfc)Co(PPh₃), and (tfc)Co(PPh₃) adsorbed on carbon is as described in example 7 above. Inks of the corrole-containing carbons were prepared by mixing 1 mg from the solid mixtures described above, with 0.2 mL isopropanol, 0.8 mL H₂O, and 10 μL of Nafion, followed by sonication for 20 min. Small quantities (5 μl) of the inks were dropped on the surface of glassy carbon electrodes (GC), followed by drying the modified electrodes for 40 min at 45° C. This process was repeated once more for each electrode and also for the ink prepared from non-modified Vulcan XC72R. The performance of the electrode prepared from the latter was checked relative to the electrodes modified by (cor)Co(PPh₃), (tpfc)Co(PPh₃), and (tfc)Co(PPh₃) by examining the electrocatalytic hydrazine oxidation.

Electrochemical hydrazine oxidation was performed using 20 mM hydrazine at pH 14 and under N₂ by ((cor)Co(PPh₃), (tpfc)Co(PPh₃), and (tfc)Co(PPh₃) catalysts adsorbed onto Vulcan XC72R carbon as the solid support. RHE and Pt wire were used as reference and counter electrodes, respectively.

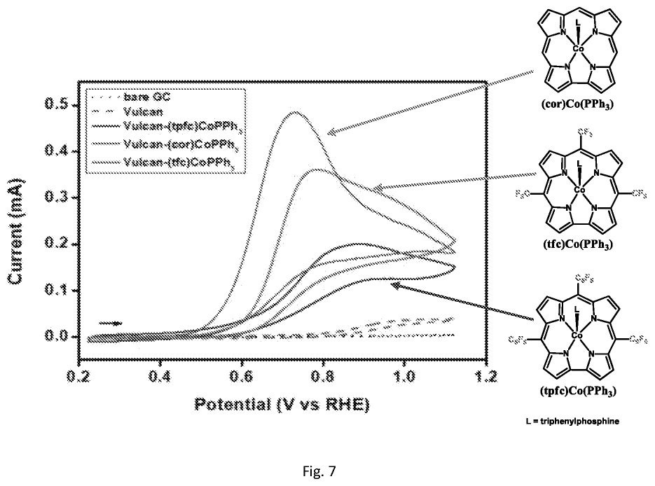

Cyclic voltammograms comparing the carbon only electrode versus the cobalt complexes are shown in FIG. 7. The best performing catalyst in terms of onset potential and catalytic current in this series was (cor)Co(PPh₃). This apparently reflects its smallest size (and hence strongest binding to the Vulcan) and least positive redox potential.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims

1. A corrole according to formula [I]; 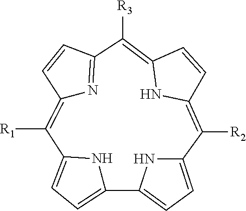
or according to Formula [II] 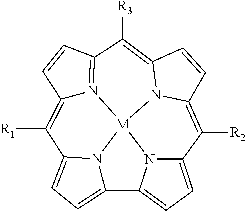
wherein R¹, R², and R³ are each independently H, —COOH, or CF₃ or a halide selected from the group consisting of F—, Cl—, Br— and I, with the proviso that R¹, R², and R³ can not all be CF₃, and when the compound is of Formula II, M is a metallic ion or an elemental ion selected from the group consisting of an elemental ion of group 13-16 in row 3 or above and boron, or a salt thereof.

2. The corrole according to claim 1, according to formula II, wherein M is selected from the group consisting of: Fe, Mn, Ga, P, Mo, Re, Co and Cu, or a salt thereof.

3. The corrole according to claim 1, wherein R¹, R², and R³ are each independently H, —COOH, or CF₃.

4. The corrole according to claim 1, wherein R¹, R² and R³ are each COOH.

5. The corrole according to claim 1, wherein R¹ and R³ are each COOH or CF₃ and R² is H.

6. The corrole according to claim 1, wherein R¹, R² and R³ are each H.

7. A method for treatment of disease comprising administration to a patient in need thereof a compound according to claim 1 wherein R¹, R², and R³ are all —COOH, or wherein at least one of them is COOH and the other(s) are either H or —CF₃, and when the compound is of Formula II, M is selected from the group consisting of Fe, Mn, P and Ga.

8. The method according to claim 7 wherein the patient suffers from a disease selected from the group consisting of: atherosclerosis, diabetes, a neurodegenerative disease, a disease associated with a oxidative/nitrosative stress and cancer.

9. A method for catalysis of a reaction comprising introducing a compound according to claim 1, wherein R¹, R², and R³ are each independently H, or CF₃ or COOH with the proviso that R¹, R², and R³ can not all be CF₃, and when the compound is of Formula II, M is selected from the group consisting of Fe, Mn, Ga, P, Mo, Re, Co and Cu.

10. The method according to claim 9 wherein R¹, R², and R³ are each independently H, or CF₃, with the proviso that R¹, R², and R³ can not all be CF₃, wherein when the compound is of Formula II, M is selected from the group consisting of Fe, Mn, Mo, Re, Co and Cu, and wherein the compound acts as an electrocatalyst.

11. The method according to claim 9 wherein R¹, R², and R³ are each independently H, —COOH, or CF₃, with the proviso that R¹, R², and R³ can not all be CF₃, wherein when the compound is of Formula II, M is selected from the group consisting of P and Ga, and wherein the compound acts as a photocatalyst.

12. The method according to claim 11 wherein the compound is adsorbed to a solid electrode or semiconducting material.

13. A method for detecting the presence of a chemical agent comprising contacting a compound of claim 1 with a chemical agent, and measuring the absorbance of the compound, wherein the compound is of formula I or II wherein R¹, R², and R³ are each independently H, —COOH, or CF₃, with the proviso that R¹, R², and R³ can not all be CF₃, wherein when the compound is of Formula II, M is selected from the group consisting of Fe and Co.

14. A photosensitizer comprising a corrole according to claim 1 complexed to a semiconducting metal oxide substrate and wherein the corrole has a structure of formula I or II wherein R¹, R², and R³ are all COOH or wherein at least one of them is COOH and the other(s) are either H or —CF₃, wherein when the compound is of Formula II, M is selected from the group consisting of P and Ga.

15. A method for imaging a subject comprising administering to the subject an amount of a corrole according to claim 1 wherein the corrole has a structure of formula II and wherein M is Mn or Fe, and applying to the subject a magnetic field, X-radiation, UV-vis light.
